(19) United States
(12) Patent Application Publication
(10) Pub. No.: US 2008/0317805 A1
(43) Pub. Date: Dec. 25, 2008

(54) LOCALLY ADMINISTRATED LOW DOSES OF CORTICOSTEROIDS

(76) Inventors: William F. McKay, Memphis, TN (US); John Myers Zanella, Cordova, TN (US); Christopher M. Hobot, Tonka Bay, MN (US)

Correspondence Address: Medtronic Spinal and Biologics;Attn: Noreen Johnson - IP Legal Department, 2600 Sofamor Danek Drive, Memphis, TN, 38132, US

(21) Appl. No.: 11/765,040

(22) Filed: Jun. 19, 2007

(51) Int. Cl. A61K 31/566; A61K 31/56; A61K 9/00; A61P 25/04

(52) U.S. Cl. 424/422; 514/169; 514/179; 514/180

(57) ABSTRACT

This invention provides for using a locally delivered low dose of a corticosteroid to treat pain caused by any inflammatory disease including sciatica, herniated disc, stenosis, mylopathy, low back pain, facet pain, osteoarthritis, rheumatoid arthritis, osteolysis, tendonitis, carpal tunnel syndrome, or tarsal tunnel syndrome. More specifically, a locally delivered low dose of a corticosteroid can be released into the epidural space, perineural space, or the foramenal space at or near the site of a patient's pain by a drug pump or a biodegradable drug depot.

### CROSS-REFERENCE TO RELATED APPLICATIONS

### FIELD OF THE INVENTION

This invention provides for using a locally delivered low dose of a corticosteroid to treat pain caused by any inflammatory disease including sciatica, herniated disc, stenosis, mylopathy, low back pain, facet pain, osteoarthritis, rheumatoid arthritis, osteolysis, tendonitis, carpal tunnel syndrome, or tarsal tunnel syndrome. More specifically, a locally delivered low dose of a corticosteroid can be released into the epidural space, perineural space, or the foramenal space at or near the site of a patient's pain by a drug pump or a biodegradable drug depot.

### BACKGROUND OF THE INVENTION

Pain is associated with many medical conditions and affects millions of Americans. The American Pain Foundation reports that over 50 million Americans suffer from chronic pain including 20% of individuals aged 60 and over who are affected by joint (arthritis or other disorders) and back pain. Furthermore, nearly 25 million Americans experience acute pain due to injuries or surgical procedures each year. The cost involved in the management of pain has been estimated at $100 billion each year. In addition to its economical burden, pain has a tremendous effect on the quality of life of affected individuals and is one of the most common causes of acute and chronic disabilities.

The human body perceives pain when body tissues, including nerve fibers, are damaged by pathogens, trauma, inflammatory conditions or noxious stimuli ranging from harmful or noxious mechanical stimuli, hot and/or cold stimuli, or chemical stimuli. Mast cells associated with damaged tissue and nerve fibers initiate the inflammation process by secreting inflammatory mediators, e.g. Tumor Necrosis Factor-alpha (TNF-a), histamine, Interleukin-1 (IL-1), IL-6, IL-8, and nerve growth factors (NGF).

These mediators cause other cells, such as monocytes, neutrophiles, and similar cells, to migrate to the trauma site. Further, these mediators also help some of the white cells, such as phagocytes, to activate their own inflammatory mediators. Inflammatory mediators, such as, NGFs secreted by damaged or irritated nerve cells and fibers have been shown to increase the number of active nerve fibers, particularly sensory fibers A and C that are involved in the transmission of nociceptive modalities. Ad fibers, a subset of the A fibers, primarily carry the fast pain, that is, the abrupt and sharp sensation type of pain quality. The C fibers are primarily responsible for transmission of the slow burning type of pain quality.

Pain and the extent of the area affected by pain can often be defined by the measure of allodynia and hyperalgesia. Allodynia is a painful response to an otherwise non-noxious stimuli. In other words, allodynia refers to pain resulting from a stimulus that ordinarily does not elicit a painful response, such as, light pressure, the movement of clothes over the skin, or the application of mild heat or cold.

Hyperalgesia is an extreme sensitivity to pain. That is, a mild noxious stimulus may be perceived as an extremely painful stimulus. In addition, hyperalgesia usually consists of primary and secondary hyperalgesic areas. Primary hyperalgesia refers to the perception of pain directly from the immediately damaged tissues. Secondary hyperalgesia refers to the perception of extreme pain sensitivity emanating from tissues immediately surrounding the primary tissue injury. Hence secondary hyperalgesia involves situations where the increased sensitization to pain has extended beyond the immediate injury and to the surrounding apparently undamaged adjacent tissues. Inflammatory mediators involved in pain are allied with various disorders that may include without limitation: osteoarthritis, rheumatoid arthritis, osteolysis, tendonitis, sciatica, herniated disc, stenosis, mylopathy, low back pain, facet pain, tendonitis, carpal tunnel syndrome, tarsal tunnel syndrome, mylopathy, etc.

In general, inflammation is a normal and essential response to any noxious stimulus and may vary from a localized to a generalized response. The inflammatory response generally follows a sequence of events that include, 1) an initial injury causing release of inflammatory mediators, such as, histamine, serotonin, leukokinins, SRS-A, lysosomal enzymes, lymphokinins, prostaglandins, etc.; 2) vasodilation, including increased vascular permeability and exudation; 3) leukocyte migration, chemotaxis, and phagocytosis; and 4) proliferation of connective tissue cells.

Corticosteroids are known in the art as being useful for treating inflammation. Corticosteroids influence all tissues of the body and produce various cellular effects. These steroids regulate carbohydrate, lipid, protein biosynthesis and metabolism, and water and electrolyte balance. Corticosteroids influencing cellular biosynthesis or metabolism are referred to as glucocorticoids while those affecting water and electrolyte balance are mineralocorticoids. Both glucocorticoids and mineralocorticoids are released from the cortex of the adrenal gland. Cortisol is the most potent glucocorticoid secreted from the adrenal gland.

For the treatment of sciatica corticosteriods have been injected into the lumbar epidural space. These steroids regulate inflammation by reducing vasodilation and the ability of phagocytes to permeate tissues. The current gold standard non-surgical treatment of sciatica is a steroid laced epidural injection. The clinical benefit of these injections is a matter of controversy. There are no set guidelines for this procedure and complications have been associated with large bolus steroid injections used to curtail neurological pain.

U.S. Pat. No. 6,468,527 (the '527 Patent) discloses a bio-based sealant composition and methods of preparation and use. The bio sealant disclosed in the '527 Patent includes combining fibrinogen and thrombin, and a corticosteroid, where the corticosteroid is used to reconstitute the thrombin from a freeze-dried state. The steroid is delivered to and held at the target area by fibrinogen's natural conversion to a fibrin clot.

U.S. Pat. No. 5,336,505 (the '505 Patent) discloses bioerodible ortho ester polymers suitable for preparing bioerodible pharmaceutical compositions such as implants, ointments, creams, gels, and the like. The '505 Patent discloses the use of specific polyorthoesters to deliver a corticosteroid.

### SUMMARY OF THE INVENTION

The present invention overcomes the drawbacks of prior art by providing a locally delivered low dose of a corticosteroid to treat pain caused by any inflammatory disease including sciatica, herniated disc, stenosis, mylopathy, low back pain, facet pain, osteoarthritis, rheumatoid arthritis, osteolysis, tendonitis, carpal tunnel syndrome, or tarsal tunnel syndrome. More specifically, a locally delivered low dose of a corticosteroid can be released into the epidural space, perineural space, or the foramenal space at or near the site of a patient's pain by a drug pump or a biodegradable drug depot.

It is an object of the invention, wherein a biodegradable drug depot comprises an implant made from a natural or synthetic biocompatible biodegradable material. Natural polymers include, but are not limited to, proteins such as albumin, collagen, gelatin, synthetic poly(aminoacids), and prolamines; glycosaminoglycans, such as hyaluronic acid and heparin; polysaccharides, such as alginates, chitosan, starch, and dextans; and other naturally occurring or chemically modified biodegradable polymers. Synthetic biocompatible biodegradable materials include but are not limited to, polyhydroxybutyric acid, poly(trimethylene carbonate), polycaprolactone (PCL), polyvalerolactone, poly(alpha-hydroxy acids), poly(lactones), poly(amino-acids), poly(anhydrides), polyketals poly(arylates), poly(orthoesters), poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer(polyactive), methacrylates, poly(N-isopropylacrylamide), PEO-PPO-PEO (pluronics), PEO-PPO-PAA copolymers, and PLGA-PEO-PLGA blends and copolymers thereof and any combinations thereof. It is another object of the invention, wherein the biodegradable drug depot is made of an implantable biocompatible biodegradable polymer comprising compositions of micro-particles, micro-spheres, capsules, gels, coatings, matrices, wafers, pills, pellets, or other pharmaceutically deliverable compositions and any combinations thereof.

It is yet another object of the invention, wherein the biodegradable drug depot is placed at or near the site of a patient's pain, which may include pain in any area within a human body resulting from inflammation, mechanical stimuli, chemical stimuli, thermal stimuli, or any combination thereof.

An embodiment of the invention includes having a biodegradable drug depot, wherein the biocompatible biodegradable polymer releases a low dose of a corticosteroid locally at or near the site of a patient's pain, which includes the epidural spaces, perineural spaces, or foramenal spaces surrounding an area of nerve irritation or the dorsal root ganglia.

Another embodiment of the invention includes having a biodegradable drug depot, wherein the biocompatible biodegradable polymer is composed of micro-particles having a particle size of about 0.1 μm to about 1000 μm, more preferably 1 μm to 200 μm, and is associated with a locally delivered low dose of a corticosteroid.

Yet another embodiment of the invention includes having a biodegradable drug depot, wherein the corticosteroid comprises dexamethasone, betamethasone, triamcinolone, triamcinolone acetonide, triamcinolone diacetate, triamcinolone hexacetonide, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, fluocinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, and triamcinolone acetonide 21-palmitate.

An object of the invention includes having a biodegradable drug depot, wherein the corticosteroid is fluocinolone and is released by the biocompatible biodegradable polymer at or near a site of a patient's pain at a rate not to exceed about 10 μg/kg/day. The rate of delivery can also range from about 1.6 μg/kg/day to about 2.56×10⁻⁴ μg/kg/day.

An object of the invention includes having a biodegradable drug depot, wherein the corticosteroid is dexamethasone and is released by the biocompatible biodegradable polymer at or near a site of a patient's pain at a rate not to exceed about 100 μg/kg/day. The rate of delivery can also range from about 20.0 μg/kg/day to about 0.001 μg/kg/day.

Yet another object of the invention includes having a biodegradable drug depot, wherein a locally delivered low dose of a corticosteroid is admixed with a biodegradable polymer for control release at or near the site of a patient's pain comprising loadings of said corticosteroid from about 0.1% to about 99% (w/w) of the polymer, more preferably about 1% to about 80%, more preferably about 1% to about 50%, most preferably about 1% to about 30%.

It is an object of the invention wherein the biodegradable drug depot has a locally delivered low dose of a corticosteroid that is associated with micro-particles including in a suitable vehicle where said locally delivered low dose of a corticosteroid is present in a weight percent relative to said micro-particle from about 0.1% to about 99% (w/w) of the polymer, more preferably about 1% to about 80%, more preferably about 1% to about 50%, most preferably about 1% to about 30%. It is an object of the invention, wherein the biodegradable drug depot further comprises a pharmaceutically acceptable excipient.

An embodiment of the invention includes having a biodegradable drug depot for treating a patient's pain, wherein the patient's pain is caused by an inflammatory disease comprising sciatica, herniated disc, stenosis, mylopathy, low back pain, facet pain, osteoarthritis, rheumatoid arthritis, osteolysis, tendonitis, carpal tunnel syndrome, or tarsal tunnel syndrome.

Yet another embodiment of the invention provides for a method of treating a patient's pain comprising the steps of: i) selection of a pain site for the local delivery of a corticosteroid; ii) placement of a biodegradable drug depot at or near the selected site and, iii) release of a locally delivered low dose of a corticosteroid at or near the selected site.

It is an object of the invention, wherein the method for treating a patient's pain includes pain caused by an inflammatory disease comprising sciatica, herniated disc, stenosis, mylopathy, low back pain, facet pain, osteoarthritis, rheumatoid arthritis, osteolysis, tendonitis, carpal tunnel syndrome, or tarsal tunnel syndrome.

Yet another embodiment of the invention includes a method of treating a patient's pain, wherein delivery of the biodegradable drug depot includes using a syringe and needle or canula to inject the depot at or near the site of a patient's pain.

It is an object of the invention wherein the method of treating pain includes delivery of the biodegradable drug depot by placing an implant having a viscous, solid, or gel form comprising micro-particles, micro-capsules, capsules, gels, coatings, matrices, wafers, pills, pellets, other pharmaceutically delivery compositions, or combinations thereof at or near said site of a patient's pain.

It is an object of the invention, wherein the method of treating a patient's pain includes delivery of the biodegradable drug depot at or near a site of a patient's pain by using an epidural needle/catheter or canula assembly or placement in the patient during surgery.

Yet another object of the invention includes a method for treating a patient's pain, wherein the site of a patient's pain includes epidural spaces, perineureal spaces, foramenal spaces, or the dorsal root ganglia.

It is an object of the invention, wherein the method of treating a patient's pain includes the corticosteroid being either fluocinolone, dexamethasone or combinations thereof.

It is an embodiment of the invention, wherein the method for treating a patient's pain is the administration of a corticosteroid being administered at a rate not to exceed 100 μg/kg/day. The rate can also range from about 100 μg/kg/day to about 1 pg/kg/day depending upon the specific activity of the compound. More specifically the corticosteroid being administered at a rate of about 50 μg/kg/day to about 100 pg/kg/day. Most specifically the corticosteroid being administered at a rate of about 30 μg/kg/day to about 500 pg/kg/day.

It is an object of the invention, wherein the method of treating a patient's pain includes having a drug pump deliver a composition comprising a locally released low dose of a corticosteroid at or near a site of a patient's pain.

Yet another embodiment of the invention includes a method for treating a patient's pain, wherein the locally released low dose of a corticosteroid is delivered by a drug pump and the composition comprising a locally released low dose of a corticosteroid includes either fluocinolone, dexamathasone, or combinations thereof.

Another embodiment of the invention includes a method for treating a patient's pain, wherein said drug pump administers locally released low dose of a corticosteroid at a rate not to exceed 100 μg/kg/day. The rate may range from about 100 μg/kg/day to about 1 pg/kg/day depending upon the specific activity of the compound at or near a site of a patient's pain. More specifically the corticosteroid being administered at a rate of about 50 μg/kg/day to about 100 pg/kg/day. Most specifically the corticosteroid being administered at a rate of about 30 μg/kg/day to about 500 pg/kg/day.

Yet another embodiment of the invention includes having a method for treating a patient's pain, wherein the patient's pain is caused by an inflammatory disease comprising sciatica, herniated disc, stenosis, mylopathy, low back pain, facet pain, osteoarthritis, rheumatoid arthritis, osteolysis, tendonitis, carpal tunnel syndrome, or tarsal tunnel syndrome.

### BRIEF DESCRIPTION OF THE DRAWINGS

FIG. 1 illustrates the effect of various doses of dexamethosone and fluocinolone on thermal paw withdrawal latency in the rat CCI model.

FIG. 2 illustrates the effect of various doses of dexamethosonse and fluocinolone on mechanical allodynia response in the rat CCI model.

### DETAILED DESCRIPTION OF THE INVENTION

Definitions

“Locally released low dose,” “locally delivered low dose,” or “locally administrated low dose” all refer to the amount of corticosteroid delivered locally to relieve pain due to inflammation, which that is less than a dose that would typically be given systemically to a patient suffering from such pain. For example, locally released low doses of corticosteroids delivered daily in human may include without limitation: cortisone: 2.5 mg/day; prednisone: 0.5 mg/day; methylprednisolone: 0.4 mg/day; triameinolone: 0.4 mg/day; betamethasone: 7.5 μg/day; dexamethasone: 7.5 μg/day; hydrocortisone: 2.0 mg/day; fluocinolone 0.3 μg/day. Locally released low doses of corticosteroids should have a dose not to exceed 100 μg/kg/day, 90 μg/kg/day, 80 μg/kg/day, 70 μg/kg/day, 60 μg/kg/day, 50 μg/kg/day, 40 μg/kg/day, 30 μg/kg/day, 20 μg/kg/day, and 10 μg/kg/day (and every integer between 100 and 10).

“Biodegradable drug depot,” “drug depot,” “the depot,” or “depot” refer to any foreign implant that a physician places into a body to release a locally delivered low dose of a corticosteroid to a patient's site of pain. The foreign implant may include without limitation: micro-particles, micro-spheres, capsules, gels, coatings, matrices, wafers, pills, fibers, pellets, or other appropriate pharmaceutical delivery compositions; all of which may, or may not, be made from a biodegradable polymer. The biodegradable polymers degrade into non-toxic residues that the body easily removes or break down or dissolve slowly and are cleared from the body intact. The polymers may be cured in-vivo or, in the alternative, ex-vivo, forming a solid matrix that incorporates the drug for controlled release to an inflammatory region. Suitable biodegradable polymers may include, without limitation natural or synthetic biocompatible biodegradable material. Natural polymers include, but are not limited to, proteins such as albumin, collagen, gelatin synthetic poly(aminoacids), and prolamines; glycosaminoglycans, such as hyaluronic acid and heparin; polysaccharides, such as alginates, chitosan, starch, and dextans; and other naturally occurring or chemically modified biodegradable polymers. Synthetic biocompatible biodegradable materials include, but are not limited to, poly(lactide-co-glycolide) (PLGA), polylactide (PLA), polyglycolide (PG), polyhydroxybutyric acid, poly(trimethylene carbonate), polycaprolactone (PCL), polyvalerolactone, poly(alpha-hydroxy acids), poly(lactones), poly(amino-acids), poly(anhydrides), polyketals poly(arylates), poly(orthoesters), poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, and PLGA-PEO-PLGA blends and copolymers thereof and any combinations thereof.

“Patient” refers to any animal, preferably a mammal, wherein mammal may include but is not limited to a dog, cat, cattle, horse, sheep, ram, llama, monkey, ape, or human.

“Drug pump” refers to any device that may be placed into the body by a physician or veterinarian, or alternatively, on the outside of the body that releases a locally delivered low dose of a corticosteroid by a mechanical or electromechanical pumping action to a inflammatory site within the body via an implanted catheter.

“Neurogenic inflammation” refers to inflammation caused by the local release of inflammation mediators by inflammatory related cells associated with irritated or damaged nerve cells or fibers and the like within the human body.

“Delivery” refers to any means used to place the drug into a patient. Such means may include without limitation, placing into a patient a biodegradable drug depot that releases the drug into a target area or attaching or inserting a drug pump in a patient that releases the drug into a target area or inserting a drug pump in to a patient that releases the drug into a target area. One of ordinary skill in the art recognizes that the biodegradable drug depot may be delivered by a wide variety of methods, e.g. placement into a drill site, injection by a syringe, catheter or canula assembly, or forceful injection by a gun type apparatus, or by placement into a surgical site in a patient during surgery. Further, various pumping machines may also deliver drugs into a target area, e.g. an osmotic pump, an interbody pump, infusion pump, implantable mini-pumps, a peristaltic pump, other pharmaceutical pumps, or a system administered locally by insertion of a catheter at or near a target site with the catheter being operably connected to a pharmaceutical delivery pump.

The terms “treatment” and “treating” a patient refer to reducing, alleviating, stopping, blocking, or preventing the symptoms of pain in a patient. For the inventions described herein, “treatment” and “treating” includes partial alleviation of symptoms as well as complete alleviation of the symptoms for a time period. The time period can be hours, days, months, or even years.

“Site of a patient's pain” refers to any area within a body causing pain, e.g. nerve root causing sciatic pain, nerve fibers growing into annular tears in discs causing back pain, a knee joint with osteoarthritis, or pain radiating from epidural or perineural spaces. The pain perceived by the patient may result from inflammatory responses, mechanical stimuli, chemical stimuli, thermal stimuli, as well as allodynia.

Alternatively, the site of a patient's pain may include any place within the body where the biodegradable drug depot or the drug pump is used in the present invention, including but is not limited to any site of injury which is causing or will cause inflammation, such as a surgical site.

Additionally, the site of a patient's pain can comprise one or multiple sites in the spine, such as between the cervical, thoracic, or lumbar vertebrae, or can comprise one or multiple sites located within the immediate area of inflamed or injured joints such as the shoulder, hip, or other joints. Implantation of the biodegradable drug depot or the drug pump can occur simultaneously with surgery to repair a fracture, remove a tumor, etc., or can be performed in individuals who experience pain, especially chronic pain, as the result of earlier trauma, injury, surgery, or other initiator of inflammation.

The site of a patient's pain also includes areas of perceived pain where the drug is deposited within a tissue, for example, a nerve root of the nervous system or a region of the brain, or in close proximity (within about 10 cm, or preferably within about 5 cm, for example) thereto.

“At or near or adjacent to the site of a patient's pain” refers to any place within the body where the biodegradable drug depot or the drug pump is used in the present invention that is immediately adjacent to damaged tissue or nerve fibers causing inflammatory pain or is within about 0.1 cm to about 10 cm from said damaged tissues or nerve cells or fibers, preferably less than 5 cm from the injury or inflammatory site.

Descriptions of various embodiments of the invention are given below. Although these embodiments are primarily intended to treat pain associated with neurogenic inflammation in or about the epidural or perinural spaces of the body, it should not be inferred that the invention is only for these uses. Any and all uses of specific words and references are simply to detail different embodiments of the present invention.

Also, any and all alterations and further modifications of the invention, as would occur to one of ordinary skill in the art, are intended to be within the scope of the invention. A non-limiting example is the prevention of osteo-diseases brought on by inflammation.

Selection of Corticosteroids and Drug Dosage

Corticosteroids associated with the present invention can be any naturally occurring or a synthetic steroid hormone. Naturally occurring corticosteroids are secreted by the adrenal cortex or generally the human body. Corticosteriods may have glucocorticoid and/or mineralocorticord activity. For the present invention non-limiting examples of corticosteroids may include: dexamethasone, betamethasone, triamcinolone, triamcinolone acetonide, triamcinolone diacetate, triamcinolone hexacetonide, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, fluocinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, and triamcinolone acetonide 21-palmitate.

The invention includes using a locally released low dose of a corticosteroid delivered daily to treat pain. A locally delivered low dose may include any daily amount of corticosteroid released by a pump or drug depot that may be less than a systemic dose that would typically be given to a patient suffering from inflammatory pain. For example, locally delivered low doses of corticosteroids delivered daily in human may include without limitation: cortisol: 2.5 mg/day; prednisone: 0.5 mg/day; methylprednisolone: 0.4 mg/day; triameinolone: 0.4 mg/day; betamethasone: 7.5 μg/day; dexamethasone: 7.5 μg/day; hydrocortisone: 2.0 mg/day; fluocinolone 0.3 μg/day. The dosage is not to exceed 100 μg/kg/day, 90 μg/kg/day, 80 μg/kg/day, 70 μg/kg/day, 60 μg/kg/day, 50 μg/kg/day, 40 μg/kg/day, 30 μg/kg/day, 20 μg/kg/day, and 10 μg/kg/day (and every integer between 100 and 10).

In certain embodiments, the dosage is provided by the biodegradable drug depot or delivered by various types of drug pumps, however the drug is to be provided at a low dose at or in close proximity to the target region of inflammation. It is desirable that the corticosteroids of the instant invention be carefully formulated for delivery in locally released low doses for the desired modulation of inflammation in a controlled and direct manner. Further, the biodegradable drug depot or a drug pump may deliver a low dose corticosteroid ranging in a continuum from a rapid or immediate release to a sustained release.

For adequate distribution and absorption in the patient, controlled release of the drug may occur at a desired site over a desired period of time. Advantageously, when the biodegradable drug depot is implanted, controlled release of the drug is capable of being directed to sites which are deep, complicated, painful or dangerous to reach by conventional means and/or otherwise inaccessible.

Polymer Depot for Control Release of Corticosteroids

Locally released low doses of corticosteroids can be delivered in a controlled and sustained manner by dispersing the steroid within a biocompatible biodegradable polymer that breaks down over time within body tissues. Further, the implant or corticosteroid may be incorporated within a protective coating that delays the release of the corticosteroid from the polymer matrix. The biocompatible biodegradable polymer should preferably degrade by hydrolysis, by either surface erosion or by bulk erosion. However, surface erosion of the polymer depot may be preferred for some applications because it ensures that release of the locally delivered low dose of the corticosteroid is not only sustained but has desirable release rates.

Many biodegradable polymers may be used to release corticosteroids to an inflammatory site. When the polymer and a corticosteroid are mixed together, the biodegradable polymer incorporates the steroid into a polymer matrix for possible sustained release of the drug at a target area within the body. The biodegradable drug depot may degrade in vivo over a period of less than about two years, where at least 50% of the drug depot dissolves anywhere from about 3 months to within about a year.

In one embodiment of the invention, the biodegradable polymer may include, without limitation, natural or synthetic biocompatible biodegradable material. Natural polymers include, but are not limited to, proteins such as albumin, collagen, gelatin synthetic poly(aminoacids), and prolamines; glycosaminoglycans, such as hyaluronic acid and heparin; polysaccharides, such as alginates, chitosan, starch, and dextans; and other naturally occurring or chemically modified biodegradable polymers. Synthetic biocompatible biodegradable materials include, but are not limited to the group comprising of, poly(lactide-co-glycolide) (PLGA), polylactide (PLA), polyglycolide (PG), polyhydroxybutyric acid, poly(trimethylene carbonate), polycaprolactone (PCL), polyvalerolactone, poly(alpha-hydroxy acids), poly(lactones), poly(amino-acids), poly(anhydrides), polyketals poly(arylates), poly(orthoesters), poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer(polyactive), methacrylates, poly(N-isopropylacrylamide), PEO-PPO-PEO (pluronics), PEO-PPO-PAA copolymers, and PLGA-PEO-PLGA blends and copolymers thereof and any combinations thereof. These polymers may be used in making controlled release or sustained release compositions disclosed herein.

Poly(d,l-lactic-co-glycolic acid) (PLGA) is commercially available from Alkermes of Cambridge, Mass. Suitable Alkermes products include 00 DL 7E, 8515 DLG 7E, 7525 DLG 7E, 6535 DLG 7E, 5050 DLG 7E (Lakeshore Biomaterials, Birmingham, Ala.); Lactel™, (Durect, Pelham, Ala.); and Resomer™ (Boeringer Ingelheim) and poly(d,l-lactic acid) (d,l-PLA), where the product's mole percent composition of lactide and glycolide are given. For example, 7525 DLG 7E have the mole percent ratios of 75% lactide and 25% glycolide. As indicated, bioerodible copolymers are available in a wide range of molecular weights and ratios of lactic to glycolic acid.

If not purchased from a supplier, then the biodegradable polymers may be prepared by the procedure set forth in U.S. Pat. No. 4,293,539 (Ludwig, et al.), the disclosure of which is hereby incorporated by reference in its entirety. Ludwig prepares such copolymers by condensation of lactic acid and glycolic acid in the presence of a readily removable polymerization catalyst (e.g., a strong acid ion-exchange resin such as Dowex HCR-W2-H).

Micro-Particles

In lieu of incorporating locally released low doses of a corticosteroid in a homogenous biodegradable drug depot, the drug depot can take the shape of small biodegradable micro-particles, that is, formulating biodegradable micro-particle that release a corticosteroid at a rate not to exceed 100 μg/kg/day, 90 μg/kg/day, 80 μg/kg/day, 70 μg/kg/day, 60 μg/kg/day, 50 μg/kg/day, 40 μg/kg/day, 30 μg/kg/day, 20 μg/kg/day, and 10 μg/kg/day (and every integer between 100 and 10). The release rate can also range from about 100 μg/kg/day to about 1 pg/kg/day depending upon the specific activity of the compound at or near a site of a patient's pain. More specifically the corticosteroid being administered at a rate of about 50 μg/kg/day to about 100 pg/kg/day. Most specifically the corticosteroid being administered at a rate of about 30 μg/kg/day to about 500 pg/kg/day. The manufacture of micro-particles or methods of making biodegradable micro-particles are known in the art. Micro-particles from any of the biodegradable polymers listed above can be made by spray drying, solvent evaporation, phase separation, fluidized bed coating or combinations thereof.

With solvent evaporation, a corticosteroid, if soluble in organic solvents, may be entrapped in the biodegradable polymer by dissolving the polymer in a volatile organic solvent, adding a locally released low dose of a corticosteroid to the organic phase, emulsifying the organic phase in water which contains a surfactant or polymer such as polyvinyl alcohol, and finally removing the solvent under vacuum to form discrete, hardened monolithic micro-particles.

Phase separation procedures entrap water-soluble agents in the polymer to prepare micro-particles. Phase separation involves coacervation of a biodegradable polymer. By addition of a nonsolvent, such as silicone oil, the polymer is then extracted from an organic solvent.

Alternatively, the micro-particles may be prepared by the process of Ramstack et al., 1995, described in published international patent application WO 95/13799, the disclosure of which is incorporated herein in its entirety. The Ramstack et al. process essentially provides for a first phase, including an active agent and a polymer, and a second phase, that are pumped through a static mixer into a quench liquid to form micro-particles containing the active agent. The first and second phases can optionally be substantially immiscible and the second phase is preferably free from solvents for the polymer and the active agent and includes an aqueous solution of an emulsifier.

In a fluidized bed coating, the drug is dissolved in an organic solvent along with the polymer. The solution is then processed, e.g., through a Wurster air suspension coating apparatus to form the final microcapsule product.

The biodegradable drug depot, can be prepared as micro-particles in a size distribution range suitable for local infiltration or injection. The diameter and shape of the micro-particles can be manipulated to modify the release characteristics. For example, smaller diameter micro-particles will have faster release rates and increased tissue penetration for locally released low dose corticosteroids. However, larger diameter micro-particles will have the opposite effect.

In addition, other particle shapes, such as, for example, cylindrical shapes, can also modify release rates of a locally released low dose corticosteroid by virtue of the increased ratio of surface area to mass inherent to such alternative geometrical shapes, relative to a spherical shape. The diameter of injectable micro-particles are in a size range from, for example, from about 1 microns to about 200 microns in diameter. In a more preferred embodiment, the micro-particles range in diameter from about 5 to about 120 microns.

Biodegradable micro-particles that release a locally delivered low dose of corticosteroids may be emulsified in suitable aqueous or non-aqueous carriers which may include, but is not limited to water, saline, pharmaceutically acceptable oils, low melting waxes, fats, lipids, liposomes and any other pharmaceutically acceptable substance that is lipophilic, substantially insoluble in water, and is biodegradable and/or eliminatable by natural processes of a patient's body. Oils of plants such as vegetables and seeds are included. Examples include oils made from corn, sesame, cannoli, soybean, castor, peanut, olive, arachis, maize, almond, flax, safflower, sunflower, rape, coconut, palm, babassu, and cottonseed oil; waxes such as carnoba wax, beeswax, and tallow; fats such as triglycerides, lipids such as fatty acids and esters, and liposomes such as red cell ghosts and phospholipid layers.

Corticosteroid Loading of Biodegradable Polymer

When a locally delivered low dose of a corticosteroid is admixed with a biodegradable polymer for a controlled release into or near the site of a patient's pain, useful loadings of said corticosteroid are from about 0.1% to about 99% (w/w) of the polymer, more preferably about 1% to about 80%, more preferably about 1% to about 50%, most preferably about 1% to about 30% of the polymer.

When the corticosteroid is included with a suitable vehicle in which microparticles comprising a locally delivered low dose of a corticosteroid are suspended, said corticosteroid is present, for example, in a weight percent relative to said corticosteroid from about 0.1% to about 99% (w/w) of the polymer, more preferably about 1% to about 80%, more preferably about 1% to about 50%, most preferably about 1% to about 30% of the polymer.

Release of the Locally Delivered Low Dosage Corticosteriod

Locally delivered low doses of corticosteroids may be incorporated into a biodegradable polymer or other controlled release formulations in a percent loading between 0.000.1% and 99.9% or more, by weight, preferably between 0.5% and 60%, or more, by weight and more preferably between 1% and 40%, or more, by weight.

It is possible to tailor the drug depot to deliver a specified loading of a locally released low dose of corticosteroids by manipulating the percent drug incorporated in the polymer and the shape of the matrix or formulation, in addition to the form of the corticosteroid and the method of production. The amount of drug released per day increases proportionately with the percentage of drug incorporated into the formulation, e.g., matrix (for example, from about 1 to about 50 to 90%). In the preferred embodiment, polymer matrices or other formulations with about 5-30% drug incorporated are utilized, although it is possible to incorporate substantially more drug, depending on the particular drug, the method used for making and loading the device, and the polymer.

As the biodegradable polymers undergo gradual bio-erosion within bodily tissues or fluids, the corticosteroid is released to the inflammatory site. The pharmacokinetic release profile of the corticosteroid by the biodegradable polymer depot may be first order, zero order, bi- or multi-phasic, to provide desired treatment of inflammatory related pain. In any pharmacokinetic event, the bio-erosion of the polymer and subsequent release of the corticosteroid may result in a controlled release of a corticosteroid from the polymer matrix. The rate of release can range from about 100 μg/kg/day to about 1 pg/kg/day depending upon the specific activity of the compound at or near a site of a patient's pain. Additional rates of release of the corticosteroid can include from approximately 95 μg/kg/day to approximately 10 pg/kg/day; approximately 90 μg/kg/day to approximately 25 pg/kg/day; approximately 85 μg/kg/day to approximately 50 pg/kg/day; approximately 80 μg/kg/day to approximately 75 pg/kg/day; approximately 75 μg/kg/day to approximately 100 pg/kg/day; approximately 70 μg/kg/day to approximately 250 pg/kg/day; approximately 65 μg/kg/day to approximately 500 pg/kg/day; approximately 60 μg/kg/day to approximately 750 pg/kg/day; approximately 55 μg/kg/day to approximately 1 ng/kg/day; approximately 50 μg/kg/day to approximately 10 ng/kg/day; approximately 45 μg/kg/day to approximately 25 ng/kg/day; approximately 40 μg/kg/day to approximately 50 ng/kg/day; approximately 35 μg/kg/day to approximately 75 ng/kg/day; approximately 30 μg/kg/day to approximately 100 ng/kg/day; approximately 25 μg/kg/day to approximately 250 ng/kg/day; approximately 20 μg/kg/day to approximately 500 ng/kg/day; and approximately 15 μg/kg/day to approximately 750 ng/kg/day. In another embodiment, the dosage of the corticosteroid is from approximately 15 μg/kg/day to approximately 50 pg/kg/day. In another embodiment, the dosage is from approximately 10 μg/kg/day to approximately 75 pg/kg/day. In another embodiment, the dosage is from approximately 5 μg/kg/day to approximately 100 pg/kg/day. In another embodiment, the dosage is from approximately 20 μg/kg/day to approximately 500 pg/kg/day. Alternatively, the rate of release can range from a rate of about 50 μg/kg/day to about 100 pg/kg/day, and even from about 30 μg/kg/day to about 500 pg/kg/day.

Excipients

The release rate of the corticosteroid from a biodegradable polymer matrix can be modulated or stabilized by adding a pharmaceutically acceptable excipient to the formulation. An excipient may include any useful ingredient added to the biodegradable polymer depot that is not a corticosteroid or a biodegradable polymer. Pharmaceutically acceptable excipients may include without limitation lactose, dextrose, sucrose, sorbitol, mannitol, starches, gum acacia, calcium phosphate, alginates, tragacanth, gelatin, calcium silicate, microcrystalline cellulose, PEG, polyvinylpyrrolidone, cellulose, water, sterile saline, syrup, and methyl cellulose. An excipient for modulating the release rate of a corticosteroid from the biodegradable drug depot may also include without limitation pore formers, pH modifiers, reducing agents, antioxidants, and free radical scavengers.

Delivery of Corticosteroid by Polymer Depot

Parenteral administration of a biodegradable composition of the invention can be mainly effected by intramuscular injection. For most body spaces, the use of a needle may be acceptable. To inject the biodegradable drug depot into an foramenal space, needles having a gauge of about 18-23 gauge are suitable. However, if a needle/catheter combination is chosen to deliver the biodegradable drug depot, then the needles through which a catheter is introduced having gauge sizes of about 16-18 gauge may be suitable.

In another embodiment, the distal end of the catheter may terminate just inside the foramenal space, for example within 3 cm of the nerve root. This embodiment may include the drug being released near the inflammatory pain site related to sciatica.

For the polymer depot of the present invention, a range of bore sizes is required for the application to various body sites (e.g., 28 to 14 gauge). This flexibility also allows for the puncturing needle encased in a plastic infusion catheter to be removable. For certain procedures that treat pain due to inflammation, thinner needles are used. Thinner needles have the same bores but are longer, and hence look thinner.

Administration of a corticosteroid via a polymer depot delivers the drug precisely to a specific area of the body. As such, one may avoid or minimize adverse events to the patient.

Delivery of Corticosteroid by a Drug Pump

A locally released low dose of corticosteroid may be delivered locally to the target area by a drug pump. The pump delivers the drug continuously and precisely to a specific area of the body. This assembly may avoid or minimizes adverse events to the patient, such as nausea or addiction to oral medications.

The controlled administration of a locally delivered low dose of corticosteroid may include, for example, an infusion pump or an implantable mini-pump inserted at the target site, or an implantable controlled release device (such as, for example, the device described in U.S. Pat. No. 6,001,386), or a sustained release delivery system (such as the system described in U.S. Pat. No. 6,007,843). The administration system may provide targeted release rates of the drug at or near the site of a patient's pain, where the pump locally releases a low dose of a corticosteroid at a rate that substantially matches a pre-selected targeted release rate. This release rate is not to exceed 100 μg/kg/day, 90 μg/kg/day, 80 μg/kg/day, 70 μg/kg/day, 60 μg/kg/day, 50 μg/kg/day, 40 μg/kg/day, 30 μg/kg/day, 20 μg/kg/day, and 10 μg/kg/day (and every integer between 100 and 10). The rate of release can range from about 100 μg/kg/day to about 1 pg/kg/day depending upon the specific activity of the compound at or near a site of a patient's pain. Additional rates of release of the corticosteroid can include from approximately 95 μg/kg/day to approximately 10 pg/kg/day; approximately 90 μg/kg/day to approximately 25 pg/kg/day; approximately 85 μg/kg/day to approximately 50 pg/kg/day; approximately 80 μg/kg/day to approximately 75 pg/kg/day; approximately 75 μg/kg/day to approximately 100 pg/kg/day; approximately 70 μg/kg/day to approximately 250 pg/kg/day; approximately 65 μg/kg/day to approximately 500 pg/kg/day; approximately 60 μg/kg/day to approximately 750 pg/kg/day; approximately 55 μg/kg/day to approximately 1 ng/kg/day; approximately 50 μg/kg/day to approximately 10 ng/kg/day; approximately 45 μg/kg/day to approximately 25 ng/kg/day; approximately 40 μg/kg/day to approximately 50 ng/kg/day; approximately 35 μg/kg/day to approximately 75 ng/kg/day; approximately 30 μg/kg/day to approximately 100 ng/kg/day; approximately 25 μg/kg/day to approximately 250 ng/kg/day; approximately 20 μg/kg/day to approximately 500 ng/kg/day; and approximately 15 μg/kg/day to approximately 750 ng/kg/day. In another embodiment, the dosage of the corticosteroid is from approximately 15 μg/kg/day to approximately 50 pg/kg/day. In another embodiment, the dosage is from approximately 10 μg/kg/day to approximately 75 pg/kg/day. In another embodiment, the dosage is from approximately 5 μg/kg/day to approximately 100 pg/kg/day. In another embodiment, the dosage is from approximately 20 μg/kg/day to approximately 500 pg/kg/day. Alternatively, the rate of release can range from a rate of about 50 μg/kg/day to about 100 pg/kg/day, and even from about 30 μg/kg/day to about 500 pg/kg/day.

One example of a suitable pump is the SynchroMed® (Medtronic, Minneapolis, Minn.) pump. This pump has three sealed chambers. The first chamber contains an electronic module and battery. The second chamber contains a peristaltic pump and drug reservoir. The third chamber contains an inert gas, which provides the pressure needed to force the drug into the peristaltic pump. To fill the pump, the drug is injected through the reservoir fill port to the expandable reservoir.

The inert gas creates pressure on the reservoir, and the pressure forces the drug through a filter and into the pump chamber. The drug is then pumped out of the device from the pump chamber and into the catheter, which will direct the drug to the target site, i.e., a location at or near the site of a patient's pain.

The rate of delivery of the drug may be controlled by a microprocessor. This allows the pump to be used to deliver similar or different amounts of the drug, at specific times, or at set intervals between deliveries, thereby controlling the release rates to correspond with the desired targeted release rates.

Alternatively, other devices suited for drug delivery can also be used to deliver a locally released low dose of a corticosteroid at or near the site of a patient's pain. Delivery devices that may be suitable for adaptation for the method of the invention include but are not limited to, for example, those devices found in U.S. Pat. No. 6,551,290 (Elsberry, et al.), which describes a medical catheter for targeted, specific drug delivery; U.S. Pat. No. 6,571,125 (Thompson), which describes an implantable medical device for controllably releasing a biologically-active agent; U.S. Pat. No. 6,594,880 (Elsberry), which describes an intraparenchymal infusion catheter system for delivering therapeutic agents to selected sites in an organism; and U.S. Pat. No. 5,752,930 (Rise, et al.), which describes an implantable catheter for infusing equal volumes of agents to spaced sites.

Additional designs which may be adaptable to be employed in the method of the present invention are provided, for example, in U.S. Pat. No. 6,913,763 to Lerner, involving a pre-programmable implantable apparatus with a feedback regulated delivery method. US patent application 2004/0106914 involving a micro-reservoir osmotic release system for controlled release of chemicals, U.S. Pat. No. 7,144,384 to Gorman et al., involving a small, light-weight device for delivering liquid medication, US 2004/0082908 involving an implantable micro-miniature infusion device, U.S. Pat. No. 6,979,351 to Forsell, involving an implantable ceramic valve pump assembly, and US 2004/0065615 involving an implantable infusion pump with a collapsible fluid chamber. Alzet® osmotic pumps (Durect Corporation, Cupertino, Calif.) are also available in a variety of sizes, pumping rates and durations suitable for use in the method of the present invention.

Based upon the condition, such as severity and duration of pain, a physician, veterinarian, or an appropriate health care professional, or the patient, based upon the condition, for example, the severity and duration of pain, may determine the local administration rate of the low dose corticosteroid at or near the site of a patient's pain. The duration of administration of the steroid, interval between locally released doses, the size of the low dose, continuity or spontaneity of dosage administration, are all appropriately determined by the physician, veterinarian, or other health care professional.

The health care professional has options in administering the drug at or near the site of a patient's pain. An effective amount of a locally released low dose of a corticosteroid and one or more additional therapeutic agents, wherein the locally administered low dose of corticosteroids and or one more additional therapeutic agents, maybe administered by a drug pump.

The drug pump's release of the locally administered low dose of a corticosteroid can (1) be localized and sustained, (2) occur over a period of at least one day to about 12 months, or (3) be continuous or periodic. Further, the health care provider has the choice of selecting a pharmaceutical composition having a targeted release rate. For example, a targeted release rate may be from about 2 weeks to about 12 months. The health care provider may vary the combinations as the patient provides feedback over the treatment course. Accordingly, the health care provider has numerous options not previously available, especially for the treatment of pain, particularly chronic pain.

### EXAMPLES

Preparation and Release Rates of 15% Fluocinolone Acetonide in PLGA Pellets

To prepare biodegradable drug depot of PLGA containing 15% fluocinolone, approximately 50 grams of 85/15 poly(D,L-lactide-co-glycolide) (PLGA) (Lakeshore Biomaterials, Birmingham, Ala.) with IV of 0.75 dL/g and molecular weight of 117 kDa, are placed in a polypropylene beaker and cooled with liquid nitrogen (approximately 200 mL) for 10 minutes. The polymer is then ground into fine particles of approximately 80 microns average diameter using an Ultra Centrifugal Mill ZM 200 (Retsch GmbH & Co., Haan, Germany). The ground polymer particles are collected and are placed in 10 cm aluminum weigh pans. The pans are placed in a vacuum oven at 35° C. under vacuum for 24 hours to remove any condensation resulting from the grinding process.

Next, 3.5 grams of polymer are weighed into an aluminum weigh pan using an analytical balance. 0.7 grams of fluocinolone acetonide (Spectrum Chemical, Gardena, Calif.) are added. The components are stirred using a spatula until the polymer and drug appear uniformly mixed. Next, 0.46 grams of polyethylene glycol methyl ether (MW 550, Sigma-Aldrich, St. Louis, Mo.) are added to the drug and polymer mixture. The components are mixed using a spatula, until the mixture appears homogeneous.

The mixture is then loaded into a HAAKE MiniLab Rheomex extruder (Model CTW5, Thermo Electron Corp, Waltham, Mass.), and is extruded through a die of 0.75 mm diameter (temperature 120° C., 25 rpm). The resulting polymeric strand is then cut into cylindrical pellets approximately 0.75 mm in length (aspect ratio=1). The cut pellets are stored in a sealed glass vial, which had been purged with dry nitrogen, until needed.

Approximately 25 mg of the pellets are weighed into each of 3 vials containing 10 mL of phosphate buffered saline, 0.5% SDS (pH 7.4). The vials are sealed and are placed in a Model C24 incubator/shaker (New Brunswick Scientific Co., Edison, N.J.) set at 37° C. and are agitated at approximately 70 RPMs. At specific time points, the elution buffer is removed and is analyzed for drug using a UV/Vis spectrophotometer at 240 nm (Model: Lambda 850, Perkin Elmer, Waltham, Mass.). The sample vials are replenished with fresh buffer and are returned to the incubator/shaker until the next time point. The cumulative drug released is plotted as a percentage of the initial drug payload.

Before 20 days, less than 10% (cumulative) of the fluocinolone elutes from the depot. On day 20, slightly more than 10% (cumulative) of the fluocinolone elutes. By day 40, approximately 15% (cumulative) of the fluocinolone elutes from the depot. By day 60, approximately 20% (cumulative) of the fluocinolone elutes from the depot.

Preparation and Release Rates of 15% Dexamethasone in PLGA Pellets

To prepare biodegradable drug depot of PLGA containing 15% dexamethasone, approximately 50 grams of 85/15 poly(D,L-lactide-co-glycolide) (PLGA) (Lakeshore Biomaterials, Birmingham, Ala.) with IV of 0.75 dL/g and molecular weight of 117 kDa, are placed in a polypropylene beaker and cooled with liquid nitrogen (approximately 200 mL) for 10 minutes. The polymer is then ground into fine particles of approximately 80 microns average diameter using an Ultra Centrifugal Mill ZM 200 (Retsch GmbH & Co., Haan, Germany). The ground polymer particles are collected and are placed in 10 cm aluminum weigh pans. The pans are placed in a vacuum oven at 35° C. under vacuum for 24 hours to remove any condensation resulting from the grinding process.

Next, 3.0 grams of polymer are weighed into an aluminum weigh pan using an analytical balance. Then 0.6 grams of dexamethasone (Spectrum Chemical, Gardena, Calif.) are added. The components are stirred using a spatula until the polymer and drug appear uniformly mixed. 0.41 grams of polyethylene glycol methyl ether (MW 550, Sigma-Aldrich, St. Louis, Mo.) are then added to the drug and polymer mixture. The components are mixed using a spatula, until the mixture appeared homogeneous.

The mixture is then loaded into a HAAKE MiniLab Rheomex extruder (Model CTW5, Thermo Electron Corp., Waltham, Mass.), and is extruded through a die of 0.75 mm diameter (temperature 120° C., 25 rpm). The resulting polymeric strand is then cut into cylindrical pellets approximately 0.75 mm in length (aspect ratio=1). The cut pellets are stored in a sealed glass vial, which had been purged with dry nitrogen, until needed.

Approximately 25 mg of the pellets are weighed into each of 3 vials containing 10 mL of phosphate buffered saline, (pH 7.4). The vials are sealed and placed in a Model C24 incubator/shaker (New Brunswick Scientific Co., Edison, N.J.) set at 37° C. and are agitated at approximately 70 RPMs. At specific time points, the elution buffer is removed and is analyzed for drug using a UV/Vis spectrophotometer at 242 nm (Model: Lambda 850, Perkin Elmer, Waltham, Mass.). The sample vials are replenished with fresh buffer and are returned to the incubator/shaker until the next time point. The cumulative drug released is plotted as a percentage of the initial drug payload.

At 2 days, about 10% (cumulative) of the drug was eluted. By 10 days, slightly less than 20% (cumulative) of the drug was eluted. By 20 days, only slightly more than 20% (cumulative) of the drug was eluted. The amount of drug eluted increased gradually to approximately 27% (cumulative) by date 60.

Dose Reduction Study of Systemically Administered Fluocinolone in the Rat Chronic Constriction Injury Model

The purpose of this study is to evaluate the efficacy of fluocinolone acetonide (Sigma Cat# F8880-25MG; Sigma Aldrich, St. Louis, Mo.), a potent corticosteroid, to reduce neuropathic pain in an animal model. This animal model involves pain-associated behaviors in male Wistar rats (300-326 g) following chronic constriction injury (CCI) induced by a procedure similar to that described by Bennett and Xie (1988). Under 2% isoflurane anesthesia, the rat's common sciatic nerve is exposed and freed from adherent tissue at mid-thigh by separating the muscle (biceps femoris) by blunt dissection. Four loose ligatures are placed 1 mm apart, using chromic gut (4-0 absorbable suture, Jorgensen Laboratories, Inc. Loveland, Colo.).

After CCI induction, each group (n=7) receive treatment via systemic injection. Vehicle control animals (Group 1) receive 1× phosphate buffered solution (PBS) intraperitoneally (IP) every three days, beginning the day of surgery (Day 0), etanercept (Group 2; 3 mg/kg) is administered IP every 3 days beginning Day 0. Animals in treatment Groups 3, 4 and 5 receive fluocinolone (0.5, 5, or 25 μg/kg) subcutaneously (SC) every day beginning Day 0.

Thermal hyperalgesia is measured using a plantar analgesia instrument (Stoelting, Wood Dale, Ill.). Prior to testing, each animal is placed on the plantar test apparatus, a clear plastic chamber, and is allowed to rest/acclimate for 15 minutes. A radiant (heat) beam stimulus is applied to the CCI paw of each animal. After paw withdrawal, an automated control interrupts both the stimulus and the timer. The heat source device is set at intensity 50, and a maximal cut-off at 15 seconds is set to prevent tissue damage. Thermal hyperalgesia paw withdrawal latency response of the injured site (right hind paw) of each animal is measured 2 days prior to CCI surgery (pre-injury baseline) on Days 7, 14, and 21 after surgery. Data from each test is analyzed by one-way ANOVA.

Mechanical allodynia is measured using von Frey monofilament test (Stoelting, Wood Dale, Ill.). The plantar surface of the CCI paw of each animal is tested as described by Chaplan et al. (1994). Each animal is placed in a suspended clear plastic chamber with a wire mesh bottom. Prior to testing, each animal is acclimated for 15 minutes. The 50% paw withdrawal threshold response is determined by sequentially increasing or decreasing the stimulus strength according to the “up-down method” of Dixon (1980).

Testing begins with a filament with a buckling weight of 2.0 g and continued through a series of filaments applied in sequence, up to about 15 g. Each filament is applied with enough pressure to cause a buckle effect. The absence of a paw lifting/withdrawal response after 5 seconds prompts the use of the filament to the next higher weight. Paw withdrawal, indicates a positive response. The testing continues for four additional measurements and is used to calculate the response threshold. Four consecutive positive responses receive a score of 0.25 g, and five consecutive negative responses (i.e., no paw withdrawal) receives a score of 15 g. The mechanical paw withdrawal threshold of each animal is measured one day prior to surgery (per-surgical baseline) and on Days 8, 15, and 22.

The 50% paw withdrawal threshold is calculated (PWT; Luo and Calcutt, 2002, Chaplan et al. 1994) using the formula 10 (Xf+?d)/10,000, where Xf is the final von Frey filament used (log units), ? is a value that analyzes the response pattern (taken from the table published by Chaplan at al., 1994), and d is the mean difference between stimuli (log units). Data is analyzed using one-way ANOVA on each test.

All animals, regardless of the treatment group, develop posture abnormalities (i.e., in walking and paw posture), following CCI of the sciatic nerve. All animals display guarding behavior (i.e., protecting the injured paw), and they place their toes together instead of spreading them apart, as normally seen in naive animals. A pronounced limp is often evident, and some animals elevated the CCI-affected paw for prolonged periods during the first few days (1-6) after surgery. The posture abnormalities are used to minimize or avoid sensory stimulation.

Tables 1A and 1B summarize the thermal paw withdrawal latencies and von Frey threshold responses, respectively, as a percentage of the pre-CCI baseline value for each behavioral test for animals treated with fluocinolone at doses of 0.5, 5, or 25 μg/kg.

TABLE 1A
Thermal Paw Withdrawal
Fluocinolone Latencies as Percent Baseline

| Treatment | | Dose Level | | |
|---|---|---|---|---|
| IP Every DAY | | 0.5 μg/kg | 5 μg/kg | 25 μg/kg |
| Day 7 | Mean | 71.6 | 79.7 | 67.4 |
| | SE | 4.6 | 4.8 | 3.9 |
| | N | 7 | 7 | 7 |
| Day 14 | Mean | 70.8 | 83.8 | 80.9 |
| | SE | 2.4 | 3.7 | 4.1 |
| | N | 5 | 7 | 7 |
| Day 21 | Mean | 63.1 | 80.2 | 80.0 |
| | SE | 4.1 | 3.0 | 5.0 |
| | N | 5 | 7 | 7 |

TABLE 1B
Von Frey Filament “Allodynia”
Fluocinolone Latencies as Percent of Baseline

| Treatment | | Dose Level | | |
|---|---|---|---|---|
| IP Every DAY | | 0.5 μg/kg | 5 μg/kg | 25 μg/kg |
| Day 8 | Mean | 45.2 | 54.8 | 76.3 |
| | SE | 9.5 | 6.8 | 20.6 |
| | N | 7 | 7 | 7 |
| Day 15 | Mean | 47.1 | 57.5 | 68.9 |
| | SE | 8.1 | 6.7 | 11.7 |
| | N | 5 | 7 | 7 |
| Day 22 | Mean | 66.2 | 55.2 | 70.3 |
| | SE | 32.8 | 11.7 | 13.2 |
| | N | 5 | 7 | 7 |

Fisher LSD tests are performed to compare each group to vehicle controls and to one another for Days 7, 14, and 21. The results reveal that across all test days, the three doses of fluocinolone produce an increase in thermal latency relative to vehicle controls (Fisher LSD, p<0.05). On Day 7, the LSD results indicate that the 5 μg/kg dose is significantly more effective than 25 μg/kg dose (Fisher LSD, p<0.05). On Days 14 and 21, both the 5 and 25 μg/kg doses are significantly more effective than the 0.5 μg/kg dose (Fisher LSD, p<0.05). Both the 5 and 25 μg/kg doses produce similar effects (Fisher LSD, p>0.05, n.s.).

The data from this study indicate that fluocinolone administered at doses of 0.5, 5, and 25 μg/kg/day significantly increases the paw withdrawal latency period following a thermal stimulus when compared to vehicle control group (ANOVA; F(3, 24)=37.21, p<0.05). In addition, fluocinolone at 5 and 25 μg/kg/day improves thermal hyperalgesia significantly greater than etanercept on all days tested (ANOVA; p<0.05). Fluocinolone at 0.5 μg/kg/day also tends to improve thermal latencies over etanercept; however, these improvements are only statistically significant on Day 7 (ANOVA; p<0.05). The data indicate that administration of fluocinolone at doses of 0.5, 5, or 25 μg/kg/day SC significantly improves (overall ANOVA) mechanical allodynia when compared to vehicle controls. In addition, the results suggest that the three doses of fluocinolone tend to improve mechanical allodynia over etanercept; however, these improvements are not statistically significant.

Daily SC administration of 25 μg/kg fluocinolone for 21 days results in a significant decrease in body weight gain (˜50 g, body weight difference by Day 22) when compared to vehicle controls. The body weight gain in this group is consistently lower than vehicle controls starting on Day 5 (˜10 g difference) and remains lower (˜50 g difference) until the end of the study. Daily SC administration of 0.5 or 5 μg/kg fluocinolone for 21 days does not have any effect on body weight gain.

In summary, the overall ANOVA indicates that fluocinolone produces a significant increase in thermal latency [F(3,24)=8.40, p<0.05]. The 0.5 μg/kg t-test results compared to etanercept, show a significant increase in latency on Day 7 (Day 7 [(12)=−3.35, p<0.05]); but not on Days 14 and 21 (Day 14 [(12)=−1.54, n.s.]; Day 21 [(12)=0.0, n.s.]). The 5 μg/kg t-test results show a significant increase in latency on all testing days: Day 7[(12)=−4.58, p<0.05]; Day 14 [(12)=−3.82, p<0.05]; and Day 21 [(12)=−2.18, p<0.05]. When comparing the mechanical thresholds of fluocinolone to etanercept, the overall ANOVA does not reveal any significant differences (F[3, 24]=+0.67, n.s.).

Pump Delivery of Fluocinolone and Dexamethasone in the Rat Chronic Constriction Injury Model

Following the above experiments, the efficacy of locally administered, low dose of fluocinolone acetonide (Sigma Cat# F8880-25MG; Lot# 043K1167, Sigma Aldrich) and dexamethasone (Sigma Aldrich) is examined in the same CCI rat model. CCI surgery is conduced as described above, and the rats are randomly assigned to 1 of 7 treatment groups (n=7).

After each CCI surgery is completed, all animals, including controls, are implanted with an Alzet® osmotic mini-pump (volume rate 0.5 μl/h) (Model 2002-Lot No. 10125-05, Durect Corp., Cupertino, Calif.) connected to a catheter (sterile catheters with suture loops) to allow for local administration of dexamethasone, fluocinolone, or PBS starting the day of injury (Day 0). The distal catheter tip is anchored with Prolene suture (4-0, non-absorbable, Ethicon, Inc., Somerville, N.J.) within the muscle in the perineural space with the catheter tip as perpendicular as possible and proximate to the sciatic nerve but without touching the nerve. The proximal end of the catheter is attached to the loaded osmotic infusion pump. The pump and catheter are tunneled up through the same incision under the skin and left in the SC space on the animal's back between the scapulae. The incision is then closed with surgical clips.

Under aseptic conditions and 2% isofluorane anesthesia, a small incision is made between the scapulae of the animal's back (directly above the pump) to exchange the pump reservoir on Day 10.

Pump reservoirs are recovered on Days 10 and 22. Residual pump volumes are collected, measured, stored at −20° C., until analyzed. Serum samples are obtained on Days 0, 5, 12, 17, and 22. Under 2-5% anesthesia, blood is taken from the retro-orbital plexus (0.5 ml of blood) from all animals. Blood is collected, allowed to coagulate in serum separator test tubes, and processed by centrifugation at 3000 rpm for 10 minutes.

Fluocinolone is administered at doses of 0.0032 ng/hour (0.02304 ng/kg/day), 0.016 ng/hour (0.1152 ng/kg/day), and 0.08 ng/hour (0.576 ng/kg/day). Dexamethasone is administered at 2.0 ng/hour (14.4 ng/kg/day), 10 ng/hour (72 ng/kg/day), and 50 ng/hour (360 ng/kg/day). 0.5 μl/hour PBS is administered as the negative control. Thermal hyperalgesia, induced and measured as described above, is measured on Days −2, 7, 14, and 21. Mechanical allodynia, induced and measured as described above, is measured on Days −1, 8, 15, and 22.

All animals, regardless of the treatment group, exhibit posture abnormalities, guarding behavior, and a pronounced limp as described above. Some animals elevate the CCI-affected paw for prolonged periods during the first few days (1-6) after surgery. These defensive posture abnormalities are seen in all groups. The observed “pain” features suggest that animals are sensitive to stimulations as a result of the CCI, and that the posture abnormalities are used to minimize or avoid sensory stimulation. In addition, none of these animals appear to have any posture abnormalities (i.e., in walking and paw posture) due to the catheter pump implant.

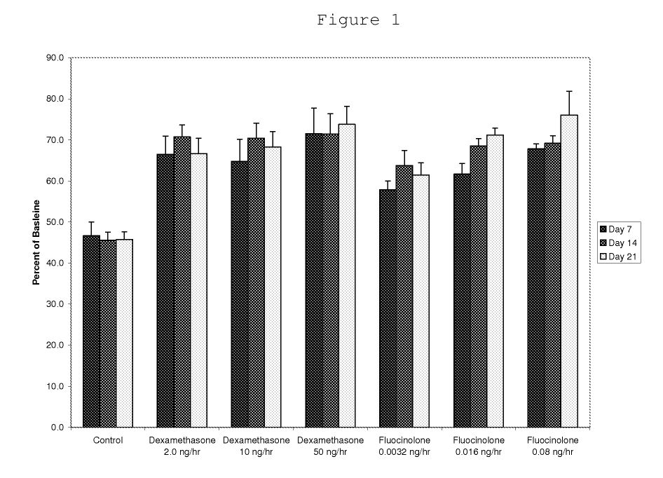

The results of the thermal paw withdrawal latency tests are disclosed in FIG. 1. As is evident from this figure, 2.0, 10, and 50 ng/hour of dexamethasone, for all three days tested, increases time until withdrawal compared to PBS. Similarly, fluocinolone at dosages of 0.0032, 0.016, and 0.08 ng/hour produce increases in the thermal paw withdrawal latency tests compared to PBS. The results for both drugs are statistically significant (p<0.05).

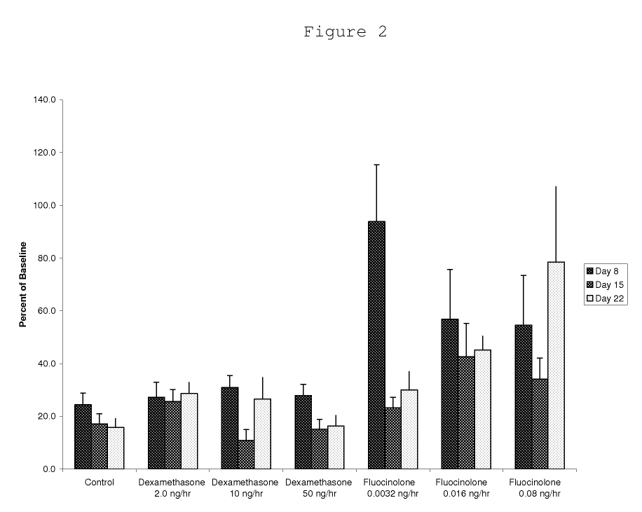

The results of the von Frey threshold response tests are disclosed in FIG. 2. For this test, all three dosages of dexamethasone provide increases in the mechanical threshold for the rats, except for 10 ng/hour at Day 15, 50 ng/hour at Day 15, and 50 ng/hour at Day 22. In addition, all three dosages of fluocinolone provide increases in the mechanical threshold for the rats for each day tested. However, the results of these tests are not statistically significant.

Although the invention herein has been described with reference to particular embodiments, it is to be understood that these embodiments are merely illustrative of the principles and applications of the present invention. For example, a person of ordinary skill in the art will appreciate that the concepts of the DNA constructs according to any embodiment of the instant invention, as well as methods and systems for delivery of these DNA constructs can be applied to other diseases, including, without limitations, diseases of the myocardium, peripheral nervous system, organs (diabetes), diseases of the spine and joints, and complex diseases such as obesity without excessive experimentation. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What we claim is:

1. A composition for the treatment of pain in a mammal comprising an biocompatible biodegradable polymer and a corticosteroid wherein said biocompatible biodegradable polymer releases said corticosteroid at a rate not to exceed 100 μg per kg of body weight of said mammal per day, wherein said corticosteroid is released at or near the site of pain in said mammal.

2. The composition of claim 1 wherein said biocompatible biodegradable polymer is selected from the group consisting of albumin, collagen, gelatin, synthetic poly(aminoacids), prolamines, glycosaminoglycans, hyaluronic acid, heparin polysaccharides, alginates, chitosan, starch, dextans, poly(lactide-co-glycolide) (PLGA), polylactide (PLA), polyglycolide (PG), polyhydroxybutyric acid, poly(trimethylene carbonate), polycaprolactone (PCL), polyvalerolactone, poly (alpha-hydroxy acids), poly(lactones), poly(amino-acids), poly(anhydrides), polyketals poly(arylates), poly(orthoesters), poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer(polyactive), methacrylates, poly(N-isopropylacrylamide), PEO-PPO-PEO (pluronics), PEO-PPO-PAA copolymers, PLGA-PEO-PLGA blends, copolymers thereof, and combinations thereof.

3. The composition of claim 1 wherein said corticosteroid is selected from the group consisting of dexamethasone, betamethasone, triamcinolone, triamcinolone acetonide, triamcinolone diacetate, triamcinolone hexacetonide, beclomethasone, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, fluocinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, clobetasol, cortisone, paramethasone, clocortolone, prednisolone 21-hemisuccinate free acid, prednisolone metasulphobenzoate, prednisolone terbutate, and triamcinolone acetonide 21-palmitate.

4. The composition of claim 1 wherein said corticosteroid is fluocinolone.

5. The composition of claim 1 wherein said corticosteroid is dexamethosone.

6. The composition of claim 1 wherein said pain is associated with a condition selected from the group consisting of an inflammatory disease, inflammation, sciatica, herniated disc, stenosis, mylopathy, low back pain, facet pain, osteoarthritis, rheumatoid arthritis, osteolysis, tendonitis, carpal tunnel syndrome, and tarsal tunnel syndrome.

7. The composition of claim 1 wherein said site of pain includes a surgical site, epidural spaces, perinureal spaces, foramenal spaces, or the dorsal root ganglia.

8. A composition for the reduction of pain in a mammal comprising an biocompatible biodegradable polymer and a corticosteroid wherein said biocompatible biodegradable polymer releases said corticosteroid at a rate not to exceed 50 μg per kg of body weight of said mammal per day, wherein said corticosteroid is released at or near the site of pain in said mammal.

9. The composition of claim 8 wherein said biocompatible biodegradable polymer is selected from the group consisting of albumin, collagen, gelatin, synthetic poly(aminoacids), prolamines, glycosaminoglycans, hyaluronic acid, heparin polysaccharides, alginates, chitosan, starch, dextans, poly(lactide-co-glycolide) (PLGA), polylactide (PLA), polyglycolide (PG), polyhydroxybutyric acid, poly(trimethylene carbonate), polycaprolactone (PCL), polyvalerolactone, poly(alpha-hydroxy acids), poly(lactones), poly(amino-acids), poly(anhydrides), polyketals poly(arylates), poly(orthoesters), poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA blends, copolymers thereof, and combinations thereof.

10. The composition of claim 8 wherein said corticosteroid is selected from the group consisting of dexamethasone, betamethasone, triamcinolone, triamcinolone acetonide, triamcinolone diacetate, triamcinolone hexacetonide, beclomethasone, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, fluocinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, clobetasol, cortisone, paramethasone, clocortolone, prednisolone 21-hemisuccinate free acid, prednisolone metasulphobenzoate, prednisolone terbutate, and triamcinolone acetonide 21-palmitate.

11. The composition of claim 8 wherein said corticosteroid is fluocinolone or dexamethosone.

12. The composition of claim 8 wherein said pain is associated with a condition selected from the group consisting of an inflammatory disease, inflammation, sciatica, herniated disc, stenosis, mylopathy, low back pain, facet pain, osteoarthritis, rheumatoid arthritis, osteolysis, tendonitis, carpal tunnel syndrome, and tarsal tunnel syndrome.

13. The composition of claim 8 wherein said site of pain includes a surgical site, epidural spaces, perinureal spaces, foramenal spaces, or the dorsal root ganglia.

14. A method for treating pain in a mammal comprising selecting a site for the local delivery of a corticosteroid placing in or on the mammal an implant for delivering the corticosteriod at the site; wherein said implant releases said corticosteriod at a rate not to exceed about 100 μg per kg of body weight of said mammal per day.

15. The method of claim 14 wherein said implant is a biodegradable drug depot comprising the corticosteroid and a biodegradable polymer.

16. The method of claim 15, wherein placing the biodegradable drug depot at the site includes using a syringe and needle, a catheter, or a canula.

17. The method of claim 14 wherein the implant comprises a drug pump, the corticosteroid, and a catheter wherein said catheter delivers said corticosteroid from said pump to said site of pain.

18. The method of claim 14, wherein the pain is associated with a condition selected from the group consisting of an inflammatory disease, inflammation, sciatica, herniated disc, stenosis, mylopathy, low back pain, facet pain, osteoarthritis, rheumatoid arthritis, osteolysis, tendonitis, carpal tunnel syndrome, and tarsal tunnel syndrome.

19. The method of claim 14 wherein the site includes a surgical site, epidural spaces, perinureal spaces, foramenal spaces, or the dorsal root ganglia.

20. The method of claim 14 wherein the corticosteroid is selected from the group consisting of dexamethasone, betamethasone, triamcinolone, triamcinolone acetonide, triamcinolone diacetate, triamcinolone hexacetonide, beclomethasone, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, fluocinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, clobetasol, cortisone, paramethasone, clocortolone, prednisolone 21-hemisuccinate free acid, prednisolone metasulphobenzoate, prednisolone terbutate, and triamcinolone acetonide 21-palmitate.

21. The method of claim 14 wherein the corticosteroid is fluocinolone or dexamethasone.

22. A method for reducing pain in a mammal comprising selecting a site for the local delivery of a corticosteroid placing in or on the mammal an implant for delivering the corticosteriod at the site; wherein said implant releases said corticosteriod at a rate not to exceed about 50 μg per kg of body weight of said mammal per day.
